(19) United States
(12) Patent Application Publication
(10) Pub. No.: US 2013/0210714 A1
(43) Pub. Date: Aug. 15, 2013

(54) PROTEIN-BOUND TRYPTOPHAN FOOD COMPOSITION

(71) Applicants: Susan P. Hudson, Toronto (CA); Craig Hudson, Toronto (CA)

(72) Inventors: Susan P. Hudson, Toronto (CA); Craig Hudson, Toronto (CA)

(21) Appl. No.: 13/818,429

(22) PCT Filed: Aug. 17, 2011
(86) PCT No.: PCT/CA2011/000939

§ 371 Date: Apr. 24, 2013

(30) Foreign Application Priority Data
Aug. 24, 2010 (CA) 2713465

(51) Int. Cl. A61K 47/48; A23L 1/305

(52) U.S. Cl. CPC A61K 47/48238; A23L 1/3051
USPC 514/5.5

(57) ABSTRACT

A composition comprising at least partially defatted meal from a plant source containing protein-bound tryptophan, which plant source has been processed to at least partially remove oil contained therein to yield said meal having a natural source of tryptophan greater than its plant source, a carbohydrate source having a high glycemic index and a physiologically-acceptable diluent or carrier therefor for inducing sleep, alleviating an anxiety disorder, depression, obsessive compulsive aggression, chronic pain or eating disorder, in a human in need thereof, wherein said composition is in the form of a food bar further comprising a puffed cereal. The composition provides improved sleep characteristics.

### FIELD OF THE INVENTION

This invention relates to compositions comprising protein-bound tryptophan for use as a food, preferably, in the form of a food bar.

### BACKGROUND OF THE INVENTION

Canadian Patent No. 2,407,562, granted in the names of Hudson, Susan P. and Hudson, Craig J. describes compositions comprising at least partially defatted meal from a plant source containing protein-bound tryptophan, preferably squash seeds, and, optionally, a carbohydrate source provided in an amount capable of facilitating transport of in vivo generated tryptophan across the blood brain barrier. Also described are dietary supplements, foods and beverages comprising the composition of the invention to induce sleep, address an anxiety disorder, depression, obsessive compulsive, aggression chronic pain and eating disorder or provide tryptophan supplementation to individuals in need thereof.

While the above compositions are satisfactory for their intended purposes there is a need to provide an aforesaid composition in a form to enhance efficacy in providing the tryptophan-protein to a person. Without being bound by theory, the inventors believe that slowing the passage of the tryptophan-bound protein in the digestion process from the stomach enhances greater absorption across the gastro-intestinal tract and will provide the enhanced efficacy of aforesaid desired biological actions.

### SUMMARY OF THE INVENTION

Accordingly, in a broad aspect the invention provides a composition comprising at least partially defatted meal from a plant source containing protein-bound tryptophan, which plant source has been processed to at least partially remove oil contained therein to yield said meal having a natural source of tryptophan greater than its plant source, a carbohydrate source having a high glycemic index and a physiologically-acceptable diluent or carrier therefor for inducing sleep, alleviating an anxiety disorder, depression, obsessive compulsive aggression, chronic pain or eating disorder, in a human in need thereof, wherein said composition is in the form of a food bar further comprising a puffed cereal.

By the term “puffed cereal” is meant the cereal is in the form of an air-containing cereal particle rather than a compact seed, grain and the like.

Preferably, the cereal is selected from corn, wheat and rice. Since the puffed cereal also is a starch that provides a high glycemic index carbohydrate in vivo the ratio of the amount of mono or di-saccharides, such as, for example, sucrose present with the tryptophan-bound protein in the composition according to the invention, may be reduced.

### BRIEF DESCRIPTION OF THE DRAWINGS

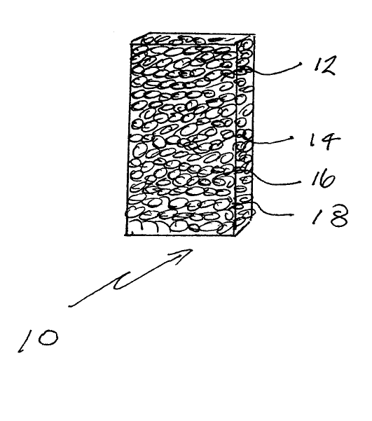

In order that the invention may be better understood preferred embodiments will now be described by way of example only wherein FIG. 1 is a diagrammatic perspective view of a food bar according to the invention.

### DETAILED DESCRIPTION OF A PREFERRED EMBODIMENT

FIG. 1 shows generally as 10, a rectangular bar having an approximate weight of 50 g. which consists of an admixture of partially defatted pumpkin seed meal 12, (33% W/W) puffed rice cereal 14, (33% W/W) sucrose 16 and caramel flavour 18, (trace) (33% (W/W).

The procedures for obtaining compositions of use in the practise of the present invention are as described in aforesaid Canadian Patent 2,407,562.

The food bar (50 gm) for use according to the invention was made according to modified general procedures described in aforesaid Canadian Patent No. 2,407,562.

Patient Report

Two human subjects reported improved sleep characteristics such as, for example, ease of falling asleep and a deeper sleep. Each subject ingested their food bar approximately 1 hour prior to retiring to bed for the evening.

Although this disclosure has described and illustrated certain preferred embodiments of the invention, it is to be understood that the invention is not restricted to those particular embodiments. Rather, the invention includes all embodiments which are functional or mechanical equivalence of the specific embodiments and features that have been described and illustrated.

1. A composition comprising at least partially defatted meal from a plant source containing protein-bound tryptophan, which plant source has been processed to at least partially remove oil contained therein to yield said meal having a natural source of tryptophan greater than its plant source, a carbohydrate source having a high glycemic index and a physiologically-acceptable diluent or carrier therefor composition is in the form of a food bar further comprising a puffed cereal.

2. A composition as claimed in claim 1 in the form of a food bar comprising partially defatted pumpkin seed flour, puffed cereal and sucrose.

3. A composition as claimed in claim 2 comprising partially defatted pumpkin seed flour (20-40% W/W), puffed rice (20-40% W/W), and sucrose (20-40% W/W).

4. Use of a composition as claimed in claim 1 for inducing sleep, or alleviating an anxiety disorder, depression, obsessive compulsive aggression, chronic pain or eating disorder, in a human in need thereof.

5. Use of a composition as claimed in claim 4 in the form of a food bar, comprising partially defatted pumpkin seed flour (20-40% W/W), puffed rice (20-40% W/W), and sucrose (20-40% W/W).
